(19) United States
(12) Patent Application Publication
(10) Pub. No.: US 2026/0124137 A1
(43) Pub. Date: May 7, 2026

(54) BILATERAL HETEROGENEOUS POLYMER-BASED ARTIFICIAL INTRAOCULAR LENS, PREPARATION PROCESS, AND USE FOR ANTIFOULING, ANTI-CELL MIGRATION, ANTI-INFLAMMATION, AND ANTI-POSTERIOR CAPSULE OPACIFICATION

(71) Applicant: National Tsing Hua University, Hsinchu (TW)

(72) Inventors: Tzu-Wei Wang, Hsinchu (TW); Cheng-Jui Yang, Hsinchu (TW)

(21) Appl. No.: 19/034,299

(22) Filed: Jan. 22, 2025

(30) Foreign Application Priority Data
Nov. 4, 2024 (TW) 113142166

(51) Int. Cl. A61K 9/00; A61K 9/06; A61K 31/717

(52) U.S. Cl. CPC A61K 9/0051; A61K 9/06; A61K 31/717

(57) ABSTRACT

The present disclosure provides a bilateral heterogeneous polymer-based artificial intraocular lens, the preparation process thereof and the use of the bilateral heterogeneous polymer-based artificial intraocular lens for antifouling, anti-cell migration, anti-inflammation and anti-posterior capsule opacification. The bilateral heterogeneous polymer-based artificial intraocular lens of the present disclosure achieves the effect of antifouling, anti-cell migration, anti-inflammation and anti-posterior capsule opacification through various efficacy experiments, and has foldable, highly transparent, antifouling bilateral heterogeneous properties.

### CROSS-REFERENCE TO RELATED APPLICATION

This application claims priority of Taiwan patent application No. 113142166, filed on Nov. 4, 2024, the content of which is incorporated herein in its entirety by reference.

### BACKGROUND OF THE INVENTION

### 1. Field of the Invention

The present invention relates to a bilateral heterogeneous polymer-based artificial intraocular lens, the preparation process, and the use of the bilateral heterogeneous polymer-based artificial intraocular lens for antifouling, anti-cell migration, anti-inflammation, and anti-posterior capsule opacification.

### 2. The Prior Art

Most artificial intraocular lenses currently on the market or used in research are synthetic polymer materials, among which hydrophobic acrylic materials are the most common. Most technologies only focus on modifying the above materials to achieve better biocompatibility.

The problems faced by hydrophilic materials are that they are easy for cells to attach and have relatively low mechanical strength, making it difficult to withstand the pressure during injection and possibly causing the extrusion of the outer capsule.

Traditionally, polyethylene glycol is often used to provide materials with antifouling properties. However, it has the disadvantages of being easily oxidized, and the hydration layer is thin. The original antifouling properties would be lost under long-term use. In the presence of metal ions, the hydroxyl group is easily converted into a carbonyl group or carboxyl group, thereby losing its hydration layer and decreasing antifouling properties.

Most artificial intraocular lenses on the market are made of hydrophobic synthetic polymer materials, which can easily cause serious foreign body reactions. At the same time, they also cause the formation of a fibrous capsule, which would isolate the artificial intraocular lens after implantation and prevent it from functioning normally. In addition, the materials currently developed cannot simultaneously meet the properties of antifouling on cell and protein and anti-cell migration. Therefore, posterior capsule opacification is still an unavoidable postoperative complication. Once residual cells adhere to the intraocular lens or migrate to the space between the intraocular lens and the posterior capsule, it affects the patients' visual field clarity.

In order to solve the above-mentioned problems, those skilled in the art urgently need to develop a novel, foldable, highly transparent, antifouling, and anti-inflammatory bilateral heterogeneous polymer-based artificial intraocular lens for preventing posterior capsule opacification after cataract surgery for the benefit of a large group of people in need thereof.

### SUMMARY OF THE INVENTION

A primary objective of the present invention is to provide a bilateral heterogeneous polymer-based artificial intraocular lens, comprising a cellulose hydrogel and dopamine, wherein the dopamine is modified on a first surface of the cellulose hydrogel.

According to an embodiment of the present invention, the cellulose hydrogel is modified with a zwitterion on a second surface relative to the first surface.

According to an embodiment of the present invention, the zwitterion is 2-methacryloyloxylethyl phosphorylcholine (MPC) or sulfobetaine methacrylate (SBMA).

According to an embodiment of the present invention, the zwitterion is modified on the second surface to form a hydration layer.

According to an embodiment of the present invention, the cellulose hydrogel is cross-linked with epichlorohydrin to form a network structure.

According to an embodiment of the present invention, the second surface is located on an intermediate area of the cellulose hydrogel.

According to an embodiment of the present invention, the first surface is located on a surrounding area of the cellulose hydrogel.

According to an embodiment of the present invention, the surrounding area is haptic and the periphery of the optic of the bilateral heterogeneous polymer-based artificial intraocular lens.

According to an embodiment of the present invention, the cellulose hydrogel is prepared through freeze-thaw cycles to improve mechanical strength and light transmittance of the cellulose hydrogel.

According to an embodiment of the present invention, the freeze-thaw (FT) cycles are 3-7 cycles.

Another objective of the present invention is to provide a method for preparing the above-mentioned bilateral heterogeneous polymer-based artificial intraocular lens, comprising the following steps: (a) preparing pre-cooled NaOH and urea solutions, followed by adding cellulose to form a cellulose solution; (b) freezing and storing the cellulose solution, and the cellulose solution is thawed, wherein the freeze-thaw (FT) cycles are repeated to obtain a transparent cellulose solution; (c) adding epichlorohydrin (ECH) dropwise into the transparent cellulose solution and putting it into a mold to form a gel, thus obtaining the cellulose hydrogel; (d) modifying the first surface of the cellulose hydrogel with dopamine; and (e) modifying a second surface of the cellulose hydrogel with a zwitterion.

According to an embodiment of the present invention, the mold has a shape of an intraocular lens and thickens the connection between the haptic and the optic.

Another objective of the present invention is to provide a method for antifouling, anti-cell migration, anti-inflammation, and anti-posterior capsule opacification, comprising using the above-mentioned bilateral heterogeneous polymer-based artificial intraocular lens to a subject in need thereof.

According to an embodiment of the present invention, the bilateral heterogeneous polymer-based artificial intraocular lens has foldable and transparent properties.

In summary, the present invention has successfully developed a foldable zwitterionic-modified hydrogel as an intraocular lens substitute through the results illustrated in the following examples. The present invention mimics commercial artificial intraocular lens (IOL) appearance and performs excellent foldability for easy insertion; exhibits superior transparency and appropriate mechanical properties to minimize the pressure exerted on the capsule; possesses high biocompatibility, hydrophilicity, and can substantially mitigate the inflammatory reaction, providing remarkable uveal biocompatibility; resists protein adsorption and prevents cell adhesion, thus achieving capsular biocompatibility to decrease the tendency of posterior capsule opacification. In addition, catechol groups oriented from dopamine promote the stable attachment between the hydrogel and the posterior capsule to inhibit the centripetal migration of lens epithelial cells.

The present invention has the following characteristics: (1) Using a self-designed mold, the natural polymer solution can be dropped into the mold to conform to the shape of the artificial intraocular lens after molding. The connection between the haptic and the optic is thickened to avoid breakage after implantation. (2) The ice crystals produced by repeated freeze-thaw cycles promote the accumulation of cellulose in the ice crystal grain boundaries. When a certain number of freeze-thaw cycles is reached, after the ice crystals melt, the cellulose can still maintain an aggregated state through mutual forces (such as hydrogen bonds), improving crystallinity. The strength of cellulose hydrogel is improved, and extremely high transmittance of the hydrogel is provided. (3) In order to prevent cellulose from hydrolysis or degradation in the human body, epichlorohydrin is used to cross-link cellulose fibers, forming chemical bonds between cellulose to form a stable network structure, reducing the risk of external factors damaging the structure. (4) In the application of artificial intraocular lenses, it is necessary to avoid the interaction between the front surface of the material and cells, proteins, etc., causing them to adhere to the material and cause cell proliferation and inflammatory reactions. Therefore, the present invention modifies zwitterions on the surface of cellulose hydrogel. Through the positive and negative charge structure on the zwitterions, it can attract water molecules to form a tight and thick hydration layer, preventing biological impurities from staying on the surface of the material. In addition, zwitterions also have good biocompatibility, which can reduce the stimulation of immune cells without producing strong inflammatory reactions. (5) Because the artificial intraocular lens and the posterior capsule must be kept in close contact to prevent the migration of residual lens epithelial cells, which may cause posterior capsule opacification. The present invention uses dopamine modification to enhance the adhesion effect of the hydrogel. The catechol on the dopamine structure can form various physical and chemical bonds with the tissue without leaving any gaps between the material and the posterior capsule. In addition, the present invention only modifies dopamine on the periphery of the haptic and the back surface of the hydrogel, which can avoid adverse effects on the light transmittance of the material while maintaining adhesion. (6) The development of intraocular lens needs to consider both uveal biocompatibility and external capsule biocompatibility. Therefore, the present invention modifies zwitterions and dopamine on the upper and lower surfaces of the hydrogel, respectively, and has the above functions. In the order of the process, before the ultraviolet light is irradiated, the surface of the intermediate area 1 of the hydrogel would be modified with zwitterions (see FIG. 1). After the grafting is successful, the central area of the surface is masked, and then Schiff base reaction is used to modify the dopamine around the hydrogel and the haptic to obtain the hydrogel with different properties in different areas.

The embodiments of the present invention will be further described below. The following examples are used to illustrate the present invention and are not intended to limit the scope of the present invention. Anyone skilled in the art can make some changes and modifications without departing from the spirit and scope of the present invention. Therefore, the scope of the present invention shall be defined by the appended claims.

### BRIEF DESCRIPTION OF THE DRAWINGS

The following drawings form part of the present specification. They are included here to further demonstrate some aspects of the present invention, which can be better understood by referencing one or more of these drawings in combination with the detailed description of the embodiments presented herein.

FIG. 1 is an overall schematic diagram of the present invention.

FIG. 2 shows surface modifications of zwitterions, in which MPC represents 2-methacryloyloxylethyl phosphorylcholine, SBMA represents sulfobetaine methacrylate, MPC-g-cellulose represents implanted MPC grafted cellulose, SBMA-g-cellulose represents implanted SBMA grafted cellulose.

FIG. 3 shows surface modifications of dopamine.

FIGS. 4A-4C demonstrate the foldability of the artificial intraocular lens. To mimic the appearance of commercial artificial intraocular lenses, we have designed our hydrogel prototype to exhibit a foldable property that allows for easy insertion and implantation during surgery.

FIGS. 5A-5C show the mechanical strength test results of cellulose hydrogels.

FIGS. 6A-6E shows antifouling test results of cellulose hydrogels, in which TCP represents tissue culture plate.

FIGS. 7A and 7B show the effect of dopamine on promoting adhesion between cellulose hydrogel and posterior capsule, in which IOL represents artificial intraocular lens, MPC represents 2-methacryloyloxylethyl phosphorylcholine, SBMA represents sulfobetaine methacrylate, MPC-g-cellulose represents implanted MPC grafted cellulose, SBMA-g-cellulose represents implanted SBMA grafted cellulose.

FIG. 8A shows the degradation curve of hydrogel, in which PEG represents polyethylene glycol, MPC represents 2-methacryloyloxylethyl phosphorylcholine, SBMA represents sulfobetaine methacrylate, MPC-g-cellulose represents implanted MPC grafted cellulose, SBMA-g-cellulose represents implanted SBMA grafted cellulose, PEG-g-cellulose represents implanted PEG grafted cellulose.

FIG. 8B shows observing the toxicity of hydrogels through cytotoxicity test reagent (Cell Counting Kit-8), in which TCP represents tissue culture plate, MPC represents 2-methacryloyloxylethyl phosphorylcholine, SBMA represents sulfobetaine methacrylate, MPC-g-cellulose represents implanted MPC grafted cellulose, SBMA-g-cellulose represents implanted SBMA grafted cellulose, PEG-g-cellulose represents implanted PEG grafted cellulose.

FIGS. 8C and 8D respectively select two pro-inflammatory factors, interleukin 1β (IL-1β) and tumor necrosis factor α (TNF-α) for identification, in which LPS represents lipopolysaccharide, MPC represents 2-methacryloyloxylethyl phosphorylcholine, SBMA represents sulfobetaine methacrylate, MPC-g-cellulose represents implanted MPC grafted cellulose, SBMA-g-cellulose represents implanted SBMA grafted cellulose, PEG-g-cellulose represents implanted PEG grafted cellulose.

FIG. 9 shows the use of a slit lamp to observe whether there is turbidity in the intraocular lens. 35 days after surgery, it can be found that only the zwitterion modified hydrogel can maintain its original transparency. The other groups (positive control, cellulose, PEG-g-cellulose) all have turbidity. The negative control group is a control group in which the intraocular lens is removed, but nothing is implanted. IOL represents artificial intraocular lens.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments enable those skilled in the art to practice the present disclosure. It is understood that other embodiments may be used and that changes can be made to the embodiments without departing from the scope of the present invention. The following description is, therefore, not to be considered as limiting the scope of the present invention.

### Definition

As used herein, the data provided represent experimental values varying within a range of ±20%, preferably within ±10%, and most preferably within ±5%.

Unless otherwise stated in the context, “a”, “the” and similar terms used in the specification (especially in the following claims) should be understood as including singular and plural forms.

The present invention is a foldable, highly transparent, antifouling bilateral heterogeneous natural polymer-based artificial intraocular lens developed to break through the difficulties faced by existing artificial intraocular lenses. The mechanical strength and light transmittance of the artificial intraocular lens are improved through freeze-thaw cycles, and different molecules are modified on the front and rear hydrogel surfaces to effectively achieve antifouling and anti-cell migration effects, avoiding the occurrence of posterior capsule opacification after cataract surgery.

The present invention can be applied to artificial intraocular lenses, urinary catheters, anti-adhesion membranes, and other materials that require antifouling on cells and proteins. The hydrogel is mainly composed of natural polymers, which can reduce inflammation and strengthen the strength and transmittance of the hydrogel through repeated freeze-thaw cycles. The hydration layer formed by zwitterions on one side (the second surface) can prevent cells, bacteria, proteins, etc. from adhering. On the other side (the first surface), dopamine can strengthen the adhesion between the hydrogel and the posterior capsule tissue, preventing surrounding cells from migrating to the vicinity of the hydrogel and affecting its light transmittance and other properties. In addition, the foldable feature can also reduce the size of surgical wounds and provide surgical convenience.

In the present invention, natural polymer cellulose is used as the main body of the hydrogel, which has good biocompatibility. In addition, the structure only has a combination of hydroxyl groups (—OH) and no amine groups (—NH₂) and carboxyl groups (—COOH). Compared with other natural polymers, it reduces the interaction with cells. At the same time, the cellulose hydrogel cross-linked with epichlorohydrin also has high toughness, forming a bendable and foldable artificial intraocular lens material.

The present invention uses a freeze-thaw cycle, repeated several times, using simple steps to improve the overall mechanical stability of the hydrogel and avoid the possibility of the hydrogel structure being damaged during the implantation process into the eye. At the same time, the physical bonds produced by the freeze-thaw cycle and the chemical bonds produced by a small amount of cross-linking agent enable the material to maintain its foldable properties.

The zwitterion molecule used in the present invention has a rich positive and negative structure. While producing a tight and thick hydration layer, it is not easily affected by environmental factors and its original characteristics.

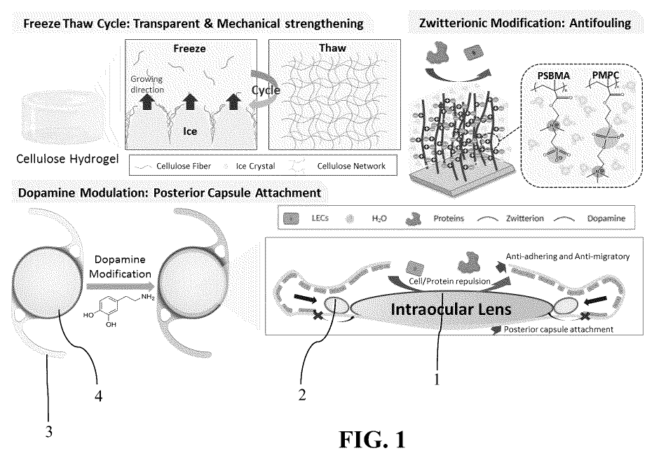

Uveal biocompatibility (affects inflammation) and external capsule biocompatibility (affects posterior capsule opacification) are two major factors that should be considered when developing and designing artificial intraocular lenses. The present invention can meet the needs simultaneously by modifying different molecules in different areas on both sides of the natural polymer cellulose hydrogel. The intermediate area of the hydrogel surface is treated with zwitterions to prevent cell attachment and proliferation. Surrounding area 2 of the surface of the hydrogel is grafted with dopamine (see FIG. 1) to promote the adhesion between the material and the tissue and prevent the gap between the hydrogel and the posterior capsule from causing cell migration, thereby minimizing the possibility of posterior capsule opacification.

The present invention is further illustrated by the following examples. These examples are provided for illustration only and are not intended to limit the scope of the present invention. The scope of the present invention is shown in the appended claims.

FIG. 1 is an overall schematic diagram of the present invention. First, a highly transparent cellulose hydrogel is produced by repeated freeze-thaw cycles, which can also enhance the mechanical stability of the hydrogel. Next, zwitterions are modified on the surface of cellulose hydrogel to prevent the adhesion of lens epithelial cells and proteins. In addition, dopamine is grafted on the haptic and periphery of the optic of the artificial intraocular lens to adhere to the posterior capsule tightly, thus hindering the migration of lens epithelial cells.

The materials used in the examples are as follows. Microcrystalline cellulose powder, urea (ACS reagent), epichlorohydrin (ECH, 99%), sodium metaperiodate (NaIO₄, 98%), sodium n-dodecyl sulfate (SDS), were obtained from Alfa Aesar (UK). Polyethylene glycol 2,000 (PEG, M_{w}=2 kDa), [2-(Methacryloyloxy) ethyl]dimethyl-(3-sulfopropyl) ammonium hydroxide (SBMA, M_{w}=279.35), 2-Methacryloyloxyethyl phosphorylcholine (<100 ppm MEHQ, MPC, M_{w}=295.27), dopamine hydrochloride, iron (II) sulfate heptahydrate (FeSO₄·7H₂O), zinc sulfate heptahydrate (ZnSO₄·7H₂O), fibronectin (from human plasma liquid) and lipopolysaccharides (from Escherichia coli, LPS) were purchased from Sigma-Aldrich (USA). Sodium hydroxide (Pellets GR, 96%, NaOH) was received from Echo (Taiwan). Ethylene glycol (99%) was purchased from J. T. Baker (USA). Bovine Serum Albumin (BSA) was supplied by Gibco (USA). Vitronectin (Recombinant Human Protein, Truncated) was obtained from Thermo Fisher Scientific (USA).

The preparation process of antifouling cellulose hydrogels is as follows. The pre-cooled NaOH/Urea solution (10 ml deionized water with 0.6 g NaOH and 0.4 g Urea) was prepared to add cellulose powder (4% w/v) at −20° C. The solution was then vigorously stirred for 1 hr at room temperature. Afterward, the FT approach was applied to improve the solubility of cellulose. The cellulose solution was stored at −80° C. for 24 hr and then thawed at room temperature for 1 hr. Repeated FT cycles were performed at different times until the transparent cellulose solution was obtained. In case of the degradation of cellulose hydrogel, cellulose fibers were united by the colorless crosslinker ECH after FT cycles. The epichlorohydrin (ECH) (8 vol. %) was added dropwise into cellulose solution, stirred for 1 hr, and placed into the Teflon self-designed mold. The cellulose hydrogel was acquired within 24 hr.

The fabrication of PEGylated cellulose hydrogel (PEG-g-cellulose) (comparison group) is similar to preparing pristine cellulose. PEG (1% w/v) was mixed with the cellulose into the NaOH/Urea solution. The previous solution was accompanied by desired FT cycles, then ECH was added to crosslink hydroxyl groups of cellulose and PEG. The PEGylated cellulose was obtained within 24 hr.

The design strategies regarding antifouling-zwitterions are as follows. Zwitterions are biologically inspired by phosphatidylcholine (PC) headgroups abundant in the phospholipid bilayer of cell membranes. They possess both anionic and cationic groups with overall charge neutrality.

Zwitterionic modifications were conducted by photo-initiated graft polymerization, which has been reported by the previous study (Hong, K. H., N. Liu, and G. Sun, UV-induced graft polymerization of acrylamide on cellulose by using immobilized benzophenone as a photo-initiator. European Polymer Journal, 2009. 45 (8): p. 2443-2449). The cellulose hydrogel was submerged in the 1% (w/v) zwitterionic (SBMA/MPC) aqueous solution, followed by the deoxygenation procedure with nitrogen gas. The mixture was then transferred to the water bath at 60° C. and treated with UV irradiation (wavelength of 365 nm) for 90 min, protected from light. After polymerization, the cellulose hydrogel was moved from the solution to the unused glass slide and rinsed with DI water at least three times to remove unreacted zwitterion monomer radically. Finally, the hydrogel was desiccated using nitrogen gas and air-dried for 1 hour to yield the zwitterionic cellulose hydrogel. The PEGylation, zwitterionic modification, and their grafting ratio were characterized by FT-IR (Bruker, Vertex 80v) and solid state 1H NMR (Bruker, Avance III 400), respectively.

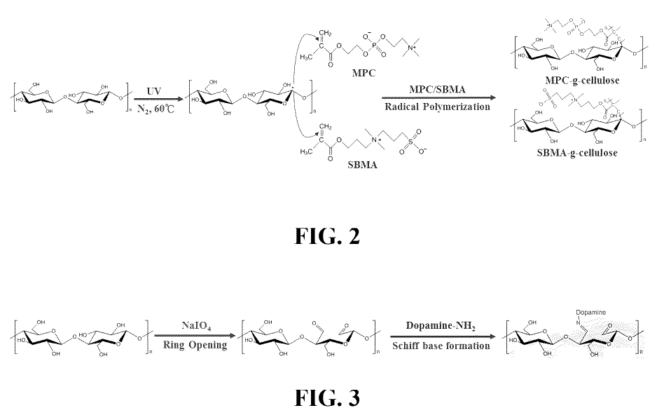

Surface modifications of zwitterions are shown in FIG. 2, in which MPC represents 2-methacryloyloxylethyl phosphorylcholine, SBMA represents sulfobetaine methacrylate, MPC-g-cellulose represents implanted MPC grafted cellulose, SBMA-g-cellulose represents implanted SBMA grafted cellulose.

The design strategies regarding bioadhesiveness-dopamine are as follows. Dopamine, inspired by mussels' adhesion to wet surfaces, is widely introduced as a coating for promoting chemical reactivity. The chemistry of the catechol in dopamine enables it to bind to organic/inorganic surfaces through non-covalent or covalent interaction, thus contributing to strong adhesion

Surface modifications of dopamine are shown in FIG. 3.

The procedure regarding localized dopamine immobilization and artificial intraocular lens (IOL) hydrogel formation is as follows. Dialdehyde cellulose (Cellulose-ALD) was produced by blending 0.4 g cellulose powder with 0.264 g NaIO4 in 20 ml of DI water to create aldehyde groups in cellulose, stirring under darkness for 24 hr. After the ring-opening reaction, 5 ml ethylene glycol was added to stop the reaction. To remove residual NaIO₄ and ethylene glycol, the mixture was purified with centrifugation by DI water for three times. Then, the obtained cellulose-ALD powder was stored at 4° C.

Dopamine-g-cellulose was accomplished through Schiff's base formation between the aldehyde groups of cellulose and the primary amine groups of dopamine. In brief, 30 mg of dopamine was dissolved in 5 ml of DI water and slowly altered the pH value to 7 to prevent the rapid oxidation of dopamine in the basic environment (pH=8.5) (Zhou, P., et al., Rapidly-deposited polydopamine coating via high temperature and vigorous stirring: formation, characterization, and biofunctional evaluation. PLOS One, 2014. 9 (11): p. e113087). Then, 100 mg of cellulose-ALD was added into the above solution and stirred at 40° C. for 4 hr. The resulting solution was centrifugated with DI water for three times to remove the unanchored dopamine. Finally, the sample was stored at −20° C. for further use. The oxidized cellulose and dopamine immobilization on cellulose-ALD was verified with FT-IR. To combine the antifouling and adhesive properties in a single material, the antifouling hydrogel was prepared in advance, and the dopamine-g-cellulose solution was poured to cover the haptic 3 and periphery of optic 4 (see FIG. 1) while blocking the center of the hydrogel. Thus, the artificial intraocular lens (IOL) hydrogel was successfully synthesized.

The procedure regarding surface characterizations is as follows. The existence of PEG and zwitterions on the cellulose hydrogel surface was confirmed through X-ray photoelectron spectroscopy (ULVAC-PHI PHI 5000 Versaprobe II) with Al Kα source for elemental composition analysis. Samples were lyophilized and cut into the area of 5×5 mm² and placed into the vacuum oven to eliminate moisture at 80° C. for 4 hr in advance. The pressure of the XPS chamber was maintained at 6.7×10⁻⁸ Pa or lower during measurement, and the spectra ranged from 0 to 1100 eV of the binding energy.

The hydrophilicity of hydrogel surfaces was examined by Contact Angle Goniometer (First Ten Angstroms-1000B). Each hydrogel sample was prepared in the 12-well tissue culture plate with enough surface area for the water droplet to contact the hydrogel steadily. A minimum of 3 drops was employed on random spots of each sample, and the average value was computed.

The design strategies regarding freeze-thaw cycles are as follows. During freezing, ice crystals will form. The cellulose nanocrystals (CNCs) will be excluded from the ice crystals and gathered in the intercrystalline grain boundaries between growing ice crystals. The physical confinement of CNCs during freezing might cause attractive forces (e.g., hydrogen bonding), resulting in CNCs aggregation into clusters and lamellar sheets, which inhibits redispersion after thawing. Different properties of hydrogels can be obtained by changing the concentration, freezing time, and number of freeze-thaw cycles.

The procedure regarding freeze-thaw effect analysis is as follows. The degree of crystallinity (CD) affected by different FT cycles was analyzed by X-ray diffractometer. The XRD profiles of lyophilized hydrogels were recorded with a diffractometer using Cu Kα radiation within the range of 10°-100° (20). The value of CD for various hydrogels was estimated by MDI Jade 6 software and was defined as:

$$CD = \frac{Area of crystalline peaks}{Area of crystalline peaks + Area of amorphous peaks} 100 \% .$$

The swelling ratio of hydrogels was evaluated under the 37° C. environment. Hydrogels were initially cleaned with delicate task wipers to remove excess moisture on the surface and then immersed into 1× phosphate buffer saline (PBS). Each hydrogel was weighed before and after immersion in PBS at specific time intervals, ie., 1, 3, 7, 14, 21, 28 days. The swelling ratio was computed by the following equation:

$$Swelling ratio ( SR ) = \frac{{w}_{f}}{{w}_{i}} \times 100 \% ,$$

where Wᵢ=initial weight of hydrogels and W_{f}=final weight of hydrogels after swelling.

The procedure regarding the optical and mechanical properties of hydrogels is as follows. Microplate spectrometer (SperctraMax Plus 384) was utilized to determine the optical clarity of hydrogels. The gelation of hydrogels was conducted in the 96-well ELISA plate with 1 mm thickness, resembling the size of commercial IOLs. Before the light transmittance test, hydrogels were washed by DI water 3 times to remove residues, and the wavelength of transmittance was collected from 300-800 nm.

The compressive and adhesive properties of hydrogels were tested by Universal Testing Machine. For compressive test, hydrogels were shaped into cylinders and placed at adequate positions to observe their mechanical behaviors. The crosshead speed of the compressive process was set at 1 mm/min to attain stress-strain curves. In the adhesive test, hydrogels were cut into 1 cm×1 cm sizes and secured between two eye tissues collected from New Zealand White rabbits. Lap shear strength was measured by stretching the ends of both tissues and the above data was analyzed by Bluehill 3 software.

The degradation behavior of hydrogels is as follows. A volume of 1 ml of hydrogel was solidified in a cylindrical mold and subsequently transferred to a 12-well tissue culture plate. Each well was then supplemented with PBS solution and incubated for specified durations at 37° C. Following incubation, the supernatants were completely aspirated, and the hydrogels were preserved at −80° C. for subsequent lyophilization. The extent of degradation was assessed by determining the dry weight of the hydrogels.

The procedure regarding the protein adsorption test is as follows. Protein antifouling performance was evaluated by bicinchoninic acid assay (BCA). The existing proteins could reduce Cu²⁺ to Cu⁺ (biuret reaction) under an alkaline environment. Next, bicinchoninic acid would chelate to Cut ions to form complexes, resulting in the color change from green to purple. Each hydrogel was gelled in a 48-well tissue culture plate, and a solution containing 0.97 μmmol/L FeSO₄ and 0.62 μmmol/L ZnSO₄ was added to some of the hydrogels for 24 hr, while others remained without the solution. This was done to observe the variation in the antifouling effect. Subsequently, hydrogels were washed and submerged in PBS solution overnight at 37° C. before the experiment. After removing PBS solution, 200 μl of bovine serum albumin (BSA) and fibronectin solution (200 μg/ml) were assigned to each well and incubated at 37° C. for 90 min. Next, each hydrogel was rinsed with PBS 3 times and transferred to 2% (w/v) sodium dodecyl sulfate (SDS) solution followed by ultrasonication for 60 min to detach proteins. Finally, protein adsorption on hydrogels was quantified by BCA assay according to the provided protocol. Absorbance at 562 nm was determined using SpectraMax Plus 384 microplate reader.

The procedure regarding in vitro cell culture is as follows. Raw 264.7 cells obtained from Bioresource Collection and Research Center (BCRC, Taiwan) were cultured in Dulbecco's Modified Eagle Medium of high glucose (DMEM-HG; Invitrogen, USA) supplemented with 10% fetal bovine serum (FBS; Gibco, USA) and 1% penicillin/streptomycin (PS; Gibco, USA). Human lens epithelial cells (LECs) were acquired from American Type Culture Collection (ATCC, Manassas, VA, US) and were grown in Eagle's Minimum Essential Medium (EMEM, ATCC, Manassas, VA, US) added with 20% FBS and 1% PS. Both cells were incubated at 37° C. in a 5% CO₂ humidified atmosphere.

The procedure regarding biocompatibility and inflammation assessment is as follows. The biocompatibility of hydrogels was evaluated by Cell Counting kit-8 (CCK-8 Assay). Each sample was sterilized by ultraviolet light for 2 hr and soaked into EMEM at 37° C. for at least 48 hr initially to assure the hydrogel interacting with culture medium uniformly. The extract liquid of hydrogels was collected for subsequent utilization. Each well was seeded with 1×10⁴ HLECs on 96-well tissue culture plate with 100 μl of leach liquor and incubated at 37° C., 5% CO₂ for predetermined time (24 hr, 48 hr, 72 hr) to observe the cell viability. After incubation, 10 μl of CCK-8 solution (10% volume of culture medium) was added into each well and reacting with HLECs at 37° C. for 1 hr until the color changed to orange. Sample absorbance at 450 nm was measured via SpectraMax Plus 384 microplate reader. HLECs seeded on the 96 well tissue culture plate individually for 24 hr was regarded as a control group.

$$Cell viability ( \% ) = \frac{{OD}_{sampe}}{{OD}_{control}} \times 100 \%$$

The inflammatory reaction was evaluated by measuring the level of cytokines secreted from Raw 264.7 macrophages, involving interleukin-1 beta (IL-1β) and tumor necrosis factor-alpha (TNF-α), both of which can be readily stimulated by lipopolysaccharides (LPS). In this experiment, the hydrogel was supported by the Transwell insert to prevent the hydrogel from directly impacting the region for the growth of macrophages. Firstly, 5×10⁵ macrophages were seeded on the 24-well tissue culture plate with 1 ml DMEM-HG medium for 24 hr. Subsequently, 100 ng/well LPS and hydrogels were placed into each well for 1 day. Afterwards, supernatants of each group were collected and stored at −20° C. for further use. Cytokines released by macrophages were analyzed with Mouse IL-1B ELISA kit and TNF-α ELISA kit following manufacturer's instructions.

The procedure regarding cell anti-adhesion performance is as follows. HLECs was selected as an indicator for anti-adhesion performance and the level of cell attachment behavior on the hydrogel surface was validated by Qubit dsDNA BR assay kit. Each hydrogel was primarily placed into 48-well tissue culture plate and a solution containing 0.97 μmmol/L FeSO₄ and 0.62 μmmol/L ZnSO₄ was added to some of the hydrogels for 24 hr. Following that, hydrogels were rinsed with PBS solution and pre-equilibrated by PBS solution. HLECs at the density of 10⁵ cell/well were then seeded onto hydrogel surfaces with 200 μl EMEM and incubated at 37° C. for predetermined time (12 hr/24 hr). Afterwards, removing EMEM and rinsing hydrogels with PBS at least three times to eliminate non-adhesive cells. Next, each sample was immersed in papain solution to extract DNA from attached cells and incubated at 65° C. overnight. The mixture was centrifugated at 10000 rpm to separate lysates and DNA and the effect of cell antiadhesion was analyzed by Qubit 2.0 fluorometer following manufacturer's indication.

The procedure regarding in vivo animal model set-up is as follows. All animal studies were conducted at Laboratory Animals Center in National Tsing Hua University and were approved by Institutional Animal Care and Use Committee (IACUC, Approval No. 112013). Female New Zealand White rabbits weighted 2-3 kg were obtained from Livestock Research Institute, Council of Agriculture (Tainan, Taiwan). All rabbits' left eyes received phacoemulsification and intraocular lens implantation. The rabbits were anesthetized with Isoflurane inhalation (induction 3-5%). Pre-operative analgesic drug, ketoprofen, 3 mg/kg was administrated through subcutaneous injection one hour before surgery. The non-operative eye (right eye) was protected with tetracycline ointment and covered with surgical tape. Pupillary dilation of the operative eye was achieved by the instillation of 0.5% tropicamide/phenylephrine (Mydrin-P) 30 minutes before the operation. After eyelash removal and preoperative sterilization of the ocular surface using povidone iodine, the surgical protocol was initiated. Throughout the operation, the operative eye was kept moisturized by balanced salt solution (BSS) irrigation. Two clear corneal incisions were created with keratomes, containing a 2.3 mm primary incision and a 1.2 mm secondary incision. Following the injection of an ophthalmic viscoelastic device (OVD) into the anterior chamber, a central continuous curvilinear capsulorhexis was performed. Subsequently, the crystalline lens was extracted using phacoemulsification approach, and the capsular bag was refilled with OVD. The implantation of a selected intraocular lens into the capsular bag was conducted, and the residual OVD was aspirated. The clear corneal incisions were closed watertight with stromal hydration or suture with 10-0 Nylon stitches if needed. Topical irrigation of gentamicin and betamethasone solution was given immediately after the operation. The postoperative treatment regimen involved topical administration of preservative-free commercial eye drops containing levofloxacin and betamethasone. These eye drops were applied four times daily for two weeks. Five groups were carried out and discussed. (1) Positive control: replace the crystalline lens with commercialized IOL. (2) Cellulose IOL: implanted pristine cellulose IOL, (3) PEG-g-cellulose IOL: implanted PEGylated cellulose IOL, (4) MPC-g-cellulose IOL: implanted MPC grafted cellulose IOL, (5) Negative control: remove crystalline lens without implanting materials.

The procedure regarding postoperative monitoring and imaging is as follows. Intraocular pressure (IOP) of rabbits' eyes was measured. Following the implantation of IOLs for durations of first, third, seventh, fourteenth, twenty-eighth and thirty-fifth days, IOP was measured. The presence of PCO was judged with slit-lamp retroillumination through the following index (Zhang, X., et al., Drug-eluting intraocular lens with sustained bromfenac release for conquering posterior capsular opacification. Bioactive Materials, 2022. 9: p. 343-357): 0=none, no PCO obeserved; 1=mild, few impurities on the optic; 2=intermediate, PCO occupying the central of the optic; 3=conspicuous, PCO spreading to the edge of the optic; 4=serious, PCO covering the integral IOL. The eye appearance was also recorded by camera at aforementioned time. Implanted IOLs and intraocular condition images were examined by surgical microscopy at the first and the last day.

The procedure regarding statistical analysis is as follows. Data were presented with mean±standard deviation (S.D.) of the mean. One-way analysis of variance (ANOVA) was utilized for the determination of the statistical significance. N.S. indicates no significance difference, * indicates p≤0.05, ** indicates p≤0.01, *** indicates p≤0.001, and **** indicates p≤0.0001.

### Example 1

### Foldability of Artificial Intraocular Lens

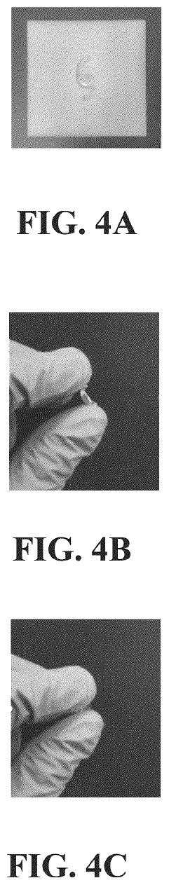

FIGS. 4A-4C demonstrate the foldability of the artificial intraocular lens. To mimic the appearance of commercial artificial intraocular lenses, we have designed our hydrogel prototype to exhibit a foldable property that allows for easy insertion and implantation during surgery. FIG. 4A shows the artificial intraocular lens hydrogel developed by the present invention and can withstand repeated bending tests. Comparing the hydrogel before bending (FIG. 4B) and after bending (FIG. 4C), the appearance remains intact and has no signs of damage. It is suitable for use in artificial intraocular lens syringes to reduce the size of surgical wounds.

### Example 2

### Mechanical Strength Test of Cellulose Hydrogels

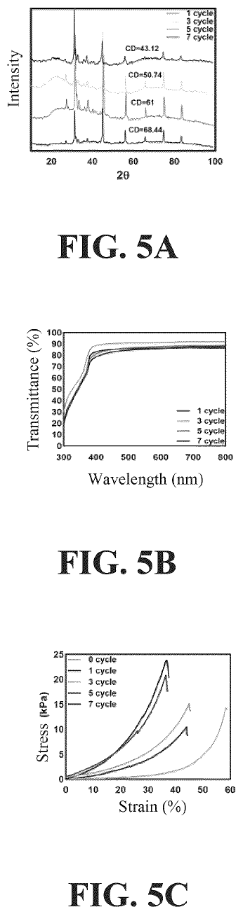

In this embodiment, the hydrogel is first subjected to 1, 3, 5, and 7 freeze-thaw cycles. It can be proved by X-ray diffractometer (XRD) that the higher the number of freeze-thaw times, the greater the crystallinity formed (FIG. 5A). After 1-7 freeze-thaw cycles, the light transmittance of the hydrogel can be maintained above 80% (FIG. 5B). The higher the crystallinity of cellulose hydrogel, the more tightly arranged the fibers are and can withstand greater pressure and provide the hydrogel with higher mechanical strength (FIG. 5C).

Preferably, the freeze-thaw (FT) cycles are 3-7 cycles to obtain the best light penetration (FIG. 5B). That is, not the more freeze-thaw cycles the better. Although increasing the number of times is beneficial to improving crystallinity and mechanical strength, it would reduce the transmittance. Therefore, it is necessary to consider both at the same time to achieve a balance.

### Example 3

### Antifouling Test of Cellulose Hydrogels

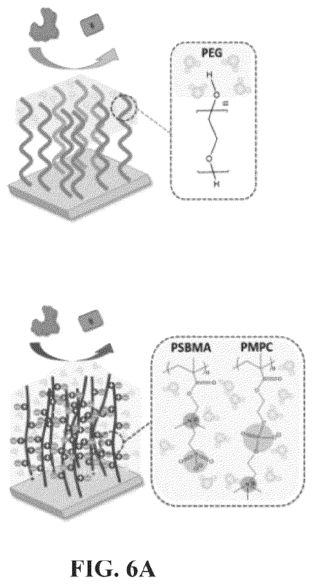

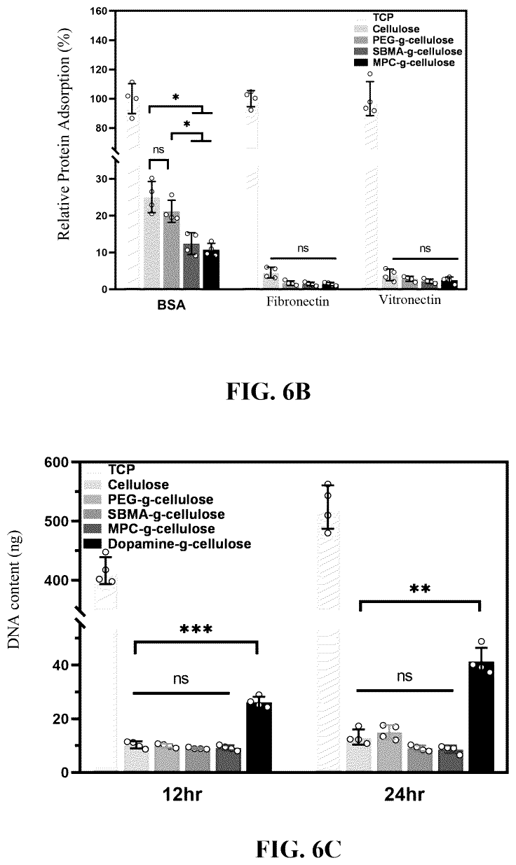

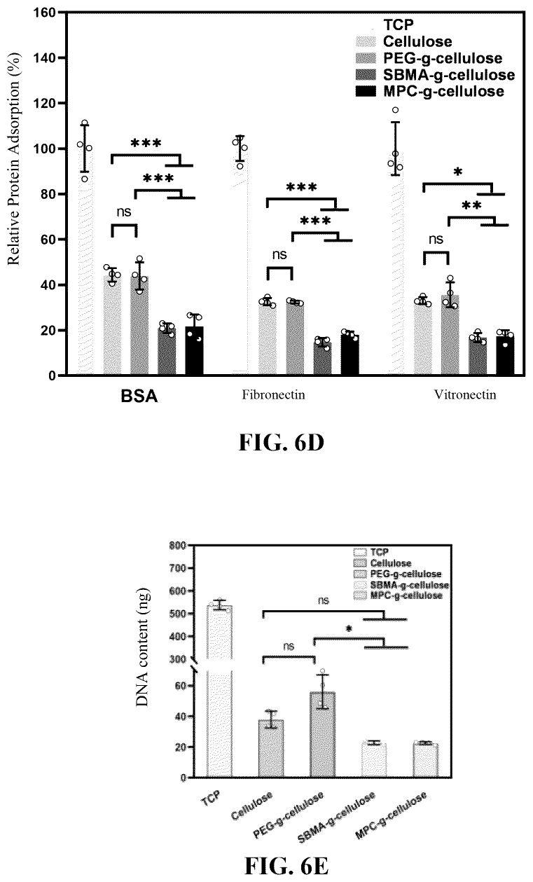

This embodiment explores the antifouling properties of hydrogels. FIG. 6A is a schematic diagram of the antifouling ability of polyethylene glycol (PEG) and zwitterions on proteins. Research results prove that cellulose hydrogels modified with zwitterions can indeed avoid the attachment of biomolecules (FIGS. 6B-6C, no transition metal treatment). In order to make a more in-depth comparison with the effect of traditional PEG modification, in this example, the modified hydrogel was pre-soaked in a solution containing trace amounts of transition metal ions to simulate the intraocular environment after artificial intraocular lens implantation. FIGS. 6D and 6E prove that the hydration layer formed by PEG would be destroyed with the participation of transition metal ions. In addition to the original incomplete hydration layer, its hydroxyl groups are easily oxidized and cannot attract water molecules. On the contrary, zwitterions always maintain a stable anti-protein adhesion effect, which can prevent proteins and cells in the eye from remaining on the surface of the hydrogel, thereby preventing excessive proliferation of lens epithelial cells.

The results of this example prove that due to the oxidation of PEG hydroxyl, then pretreated with a solution containing Fe²⁺/Zn²⁺, PEG-g-cellulose (PEGylated cellulose hydrogel) exhibits poor antifouling properties compared to zwitterion modified cellulose.

### Example 4

### Evaluation of the Effect of Dopamine on Promoting Adhesion Between Cellulose Hydrogel and Posterior Capsule

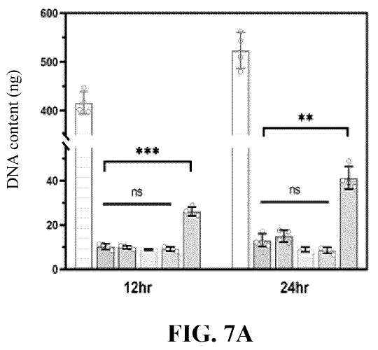

This embodiment tests whether dopamine can promote the adhesion between cellulose hydrogel and the posterior capsule. FIG. 7A shows the cell experiment. When epithelial cells were cultured on the surface of each hydrogel, it was found that compared with other groups, the number of cells in dopamine-modified hydrogels increased significantly. In addition, this example also uses animal tissue to test the adhesive properties of the hydrogel. The hydrogel is placed between the two pieces of pig skin respectively. The shear stress was measured using a universal testing machine. It was found that the dopamine-modified group was more difficult to separate from the pig skin and produced greater shear stress, indicating higher adhesion properties and more difficult to separate (FIG. 7B).

### Example 5

Assessment of Possible Situations after Hydrogel Implantation

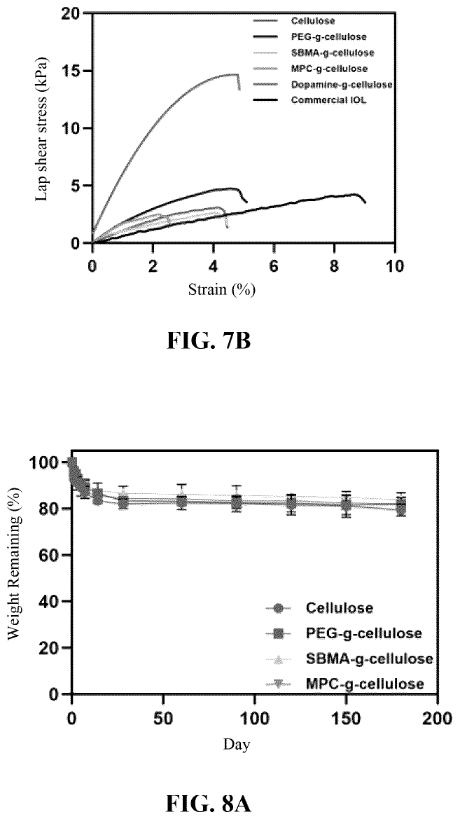

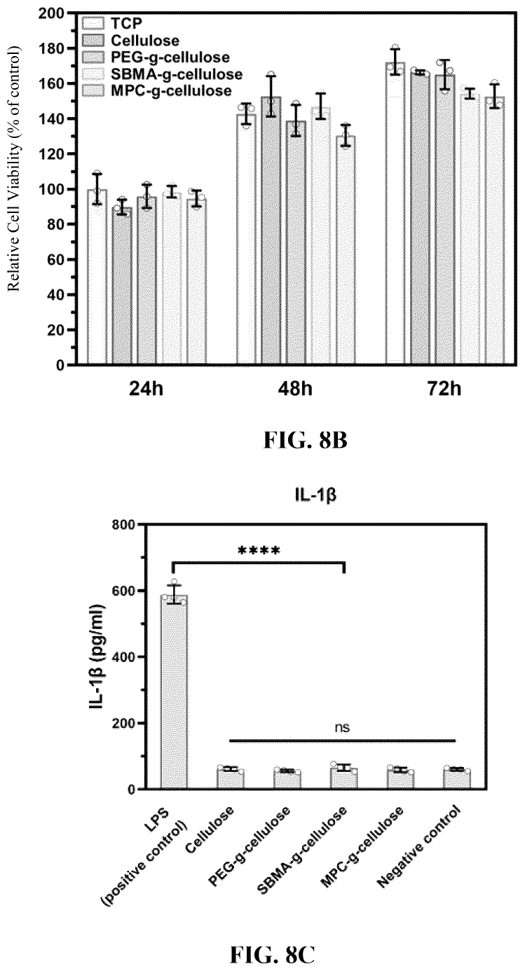

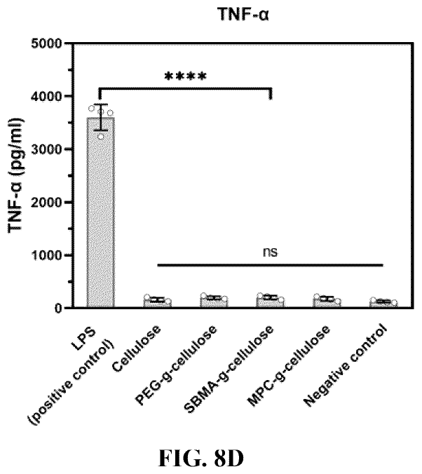

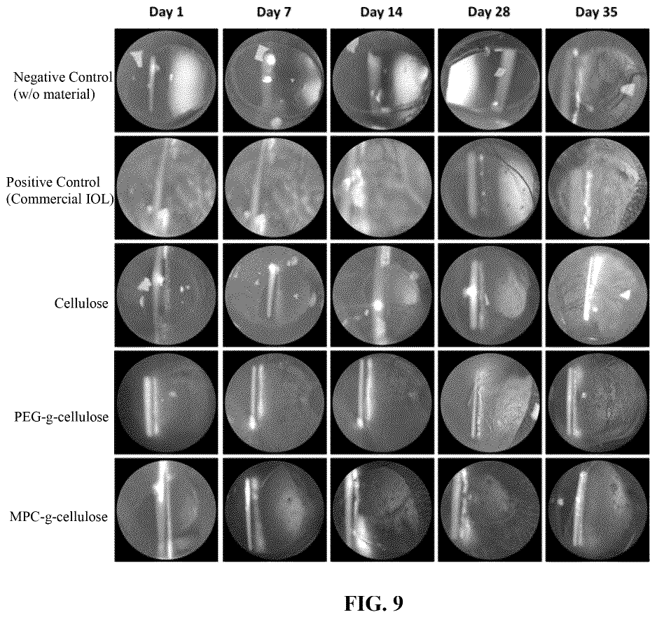

This embodiment is to evaluate the possible situation after hydrogel implantation, that is, the biocompatibility evaluation, and is tested by in vitro experiments. FIG. 8A shows the degradation curve of the hydrogel. After about 30 days, the degradation of hydrogel becomes gentle and is higher than 80%, and can persist for more than 6 months. FIG. 8B shows observing the toxicity of hydrogels through cytotoxicity test reagent (Cell Counting Kit-8). The growth of cells in each group was normal, indicating that the materials used are highly biocompatible. Finally, the inflammatory factors produced by macrophages co-cultured with hydrogels were quantified to test the degree of inflammation caused by hydrogels. FIGS. 8C and 8D, respectively, select two pro-inflammatory factors, interleukin 1B (IL-1B) and tumor necrosis factor α (TNF-α), for identification. Quantitative determination of IL-1β and TNF-α secreted by macrophages cultured on various hydrogels to assess inflammatory response. Both prove that this hydrophilic material is less likely to cause inflammation.

The results of this example prove that after modification, the cellulose hydrogel exhibits an excellent proliferation rate and lower IL-1β and TNF-α expression, indicating that it is non-toxic and does not induce inflammatory reactions.

### Example 6

Status of Artificial Intraocular Lens Hydrogel after Implantation in the Eye

In this embodiment, the eyes of New Zealand white rabbits are used as animal experimental models to observe the conditions of different artificial intraocular lens hydrogels after being implanted in the eyes. This invention removes the natural intraocular lens through phacoemulsification surgery, and then injects the hydrogel material into the rabbit eye with an artificial intraocular lens syringe. It is also compared with commercially available artificial intraocular lenses. FIG. 9 shows the use of a slit lamp to observe whether there is turbidity in the intraocular lens. 35 days after surgery, it can be found that only the zwitterion modified hydrogel can maintain its original transparency. The other groups (positive control, cellulose, PEG-g-cellulose) all have turbidity. The negative control group is a control group in which the intraocular lens is removed but nothing is implanted.

In summary, the present invention has successfully developed a foldable zwitterionic-modified hydrogel as intraocular lens substitute through the results illustrated in the above examples. The present invention mimics commercial IOL appearance and performs excellent foldability for easy insertion; exhibits superior transparency and appropriate mechanical properties in order to minimize the pressure exerted on the capsule; possesses high biocompatibility, hydrophilicity, and can substantially mitigate the inflammatory reaction, providing remarkable uveal biocompatibility; resists protein adsorption and prevents cell adhesion, thus achieving capsular biocompatibility to decrease the tendency of posterior capsule opacification. In addition, catechol groups oriented from dopamine promote the stable attachment between the hydrogel and the posterior capsule to inhibit the centripetal migration of lens epithelial cells.

The present invention has the following characteristics: (1) Cellulose-based material. Naturally-derived polymer with remarkable hydrophilicity, biocompatibility and appropriate mechanical properties compared with commercial IOL materials can decrease the incidence of glistening and substantially mitigate the inflammatory reaction. (2) Freeze thaw method. Repeated freeze-thaw cycles provide a facile physical approach to strengthen the mechanical property and achieve superior transparency resulting from the cellulose clusters and lamellar sheets formed by the aggregation of aligned cellulose via attractive forces (e.g., hydrogen bonding) to inhibit redispersion after thawing. (3) Zwitterionic modification. The tight hydration layer formed by the interaction of water molecules and zwitterions can resist protein adsorption and prevent form cell adhesion, thus decreasing the tendency of posterior capsule opacification. (4) Heterogeneous intraocular lens design. Through localized dopamine modification on the haptics and the peripheral optic, the intraocular lens can adhere to the posterior capsule firmly to inhibit the centripetal migration of lens epithelial cells while maintaining its transparency at the central optical zone.

Although the present invention has been described with reference to the preferred embodiments, it will be apparent to those skilled in the art that various modifications and changes in form and detail may be made without departing from the scope of the present invention defined by the appended claims.

What is claimed is:

1. A bilateral heterogeneous polymer-based artificial intraocular lens, comprising a cellulose hydrogel and dopamine, wherein the dopamine is modified on a first surface of the cellulose hydrogel.

2. The bilateral heterogeneous polymer-based artificial intraocular lens according to claim 1, wherein the cellulose hydrogel is modified with a zwitterion on a second surface relative to the first surface.

3. The bilateral heterogeneous polymer-based artificial intraocular lens according to claim 2, wherein the zwitterion is 2-methacryloyloxylethyl phosphorylcholine (MPC) or sulfobetaine methacrylate (SBMA).

4. The bilateral heterogeneous polymer-based artificial intraocular lens according to claim 2, wherein the zwitterion is modified on the second surface to form a hydration layer.

5. The bilateral heterogeneous polymer-based artificial intraocular lens according to claim 1, wherein the cellulose hydrogel is cross-linked with epichlorohydrin to form a network structure.

6. The bilateral heterogeneous polymer-based artificial intraocular lens according to claim 2, wherein the second surface is located on an intermediate area of the cellulose hydrogel.

7. The bilateral heterogeneous polymer-based artificial intraocular lens according to claim 6, wherein the first surface is located on a surrounding area of the cellulose hydrogel.

8. The bilateral heterogeneous polymer-based artificial intraocular lens according to claim 7, wherein the surrounding area is haptic and periphery of optic of the bilateral heterogeneous polymer-based artificial intraocular lens.

9. The bilateral heterogeneous polymer-based artificial intraocular lens according to claim 1, wherein the cellulose hydrogel is prepared through freeze-thaw cycles to improve mechanical strength and light transmittance of the cellulose hydrogel.

10. The bilateral heterogeneous polymer-based artificial intraocular lens according to claim 9, wherein the freeze-thaw (FT) cycles are 3-7 cycles.

11. A method for preparing the bilateral heterogeneous polymer-based artificial intraocular lens according to claim 1, comprising the following steps:
(a) preparing pre-cooled NaOH and urea solutions, followed by adding cellulose to form a cellulose solution;
(b) freezing and storing the cellulose solution, and the cellulose solution is thawed, wherein the freeze-thaw (FT) cycles are repeated to obtain a transparent cellulose solution;
(c) adding epichlorohydrin (ECH) dropwise into the transparent cellulose solution and putting it into a mold to form a gel, thus obtaining the cellulose hydrogel;
(d) modifying the first surface of the cellulose hydrogel with dopamine; and
(e) modifying a second surface of the cellulose hydrogel with a zwitterion.

12. The method according to claim 11, wherein the zwitterion is 2-methacryloyloxylethyl phosphorylcholine (MPC) or sulfobetaine methacrylate (SBMA).

13. The method according to claim 11, wherein the zwitterion is modified on the second surface to form a hydration layer.

14. The method according to claim 11, wherein the cellulose hydrogel is cross-linked with epichlorohydrin to form a network structure.

15. The method according to claim 11, wherein the second surface is located on an intermediate area of the cellulose hydrogel.

16. The method according to claim 11, wherein the first surface is located on a surrounding area of the cellulose hydrogel.

17. The method according to claim 16, wherein the surrounding area is haptic and periphery of optic of the bilateral heterogeneous polymer-based artificial intraocular lens.

18. The method according to claim 17, wherein the mold has shape of an intraocular lens and thickens connection between the haptic and the optic.

19. A method for antifouling, anti-cell migration, anti-inflammation, and anti-posterior capsule opacification, comprising using the bilateral heterogeneous polymer-based artificial intraocular lens according to claim 1 to a subject in need thereof.

20. The method according to claim 19, wherein the bilateral heterogeneous polymer-based artificial intraocular lens has foldable and transparent properties.
